(19) United States
(12) Patent Application Publication
(10) Pub. No.: US 2007/0116296 A1
(43) Pub. Date: May 24, 2007

(54) AUDIO PROCESSING SYSTEM AND METHOD FOR HEARING PROTECTION IN AN AMBIENT ENVIRONMENT

(75) Inventors: Kuan-Hong Hsieh, Shenzhen, Guangdong (CN); Xiao-Guang Li, Shenzhen, Guangdong (CN); Wen-Chuan Lian, Shenzhen, Guangdong (CN); Wen-Sheng Tsai, Shenzhen, Guangdong (CN); Bing Li, Shenzhen, Guangdong (CN)

Correspondence Address: NORTH AMERICA INTELLECTUAL PROPERTY CORPORATION, P.O. BOX 506, MERRIFIELD, VA, 22116, US

(73) Assignee: HON HAI PRECISION INDUSTRY CO., LTD., Taipei Hsien (TW)

(21) Appl. No.: 11/557,093

(22) Filed: Nov. 6, 2006

(30) Foreign Application Priority Data
Nov. 18, 2005 (TW) 094140638

(51) Int. Cl. A61F 11/06

(52) U.S. Cl. 381/72

(57) ABSTRACT

The present invention relates to an audio processing method for hearing protection in an ambient environment, includes steps of: sampling digital audio signals decoded to obtain a plurality of amplitude values of the digital audio signals; obtaining a default gain value; calculating an actual audio energy of the digital audio signals sampled during a predetermined period time according to the amplitude values sampled in the predetermined period time and the default gain value; collecting ambient noises to calculate an ambient noise level; obtaining a reference audio energy according to the ambient noise level; comparing the actual audio energy with the reference audio energy; and generating a hearing protection signal if the actual audio energy reaches the reference audio energy, whereby protecting users' hearing. The present invention also provides a corresponding audio processing apparatus.

### BACKGROUND OF THE INVENTION

1. Field of the Invention

The present invention relates to an audio processing apparatus and method for hearing protection in an ambient environment, especially to an audio processing apparatus and method for evaluating ambient noise level, and automatically adjusting a default gain value or emitting a prompt signal according to the ambient noise level evaluated.

2. Description of Related Art

The continuous development of new digital technologies has helped portable audio devices (such as MP3 player) become popular. When environmental noise external of the portable audio device is loud or when a favorite song is played, a user commonly increases a gain value of the portable audio device. However users all have a physiological hearing threshold, i.e., loudness discomfort level (LDL). If the user is exposed to an environment with a noise level that is larger than the user's LDL for a long time, the user's hearing may be damaged.

In order to solve the problems mentioned, there is a common gain control apparatus and method available in the market. The gain control apparatus provides a gain setting table, the gain setting table lists a plurality of gain values corresponding to each noise level. The gain control apparatus firstly senses ambient noise, and calculates an ambient noise level of the ambient noise in a predetermined time field; obtaining a predetermined gain value corresponding to the ambient noise level from the gain setting table; adjusts a gain value to the predetermined gain value. Whereby the gain value of the gain control apparatus is changeable along with the ambient noise level.

However, if a user is in an environment where the ambient noise level changes frequently, the gain control apparatus will frequently change the gain value accordingly, whereby the user will be uncomfortable. Furthermore, audio signals with different amplitudes will have different loudness at a same gain value.

Therefore, a heretofore unaddressed need exists in the industry to overcome the aforementioned deficiencies and inadequacies.

### SUMMARY OF THE INVENTION

In order to solve said problems, the present invention provides an audio processing method, apparatus and system for hearing protection while an audio processing apparatus outputs audio signals in an ambient environment. The audio processing method, apparatus and system evaluates ambient noise level, and automatically adjusts a gain value or emitting a prompt signal according to the ambient noise level evaluated.

The audio processing method includes the steps of: sampling digital audio signals decoded to obtain a plurality of amplitude values of the digital audio signals; obtaining a default gain value; calculating an actual audio energy of the digital audio signals sampled during a predetermined period time according to the amplitude values sampled in the predetermined period time and the default gain value; collecting ambient noises to calculate an ambient noise level; obtaining a reference audio energy according to the ambient noise level; comparing the actual audio energy with the reference audio energy; and generating a hearing protection signal if the actual audio energy reaches the reference audio energy.

The audio processing apparatus includes a storage unit, a processing unit, a decoding unit, a digital/analog converter, a gain control unit, a sound output device, a microphone, an analog/digital converter and a gain management unit. The storage unit stores a plurality of audio files and a default gain value. The processing unit fetches an audio file from the storage unit. The decoding unit decodes the audio file fetched to generate digital audio signals. The digital/analog converter converts the digital audio signals to analog audio signals. The gain control unit amplifies the analog audio signals converted according to the default gain value stored in the storage unit. The sound output device outputs sound corresponding to the analog audio signals amplified. The microphone collects ambient noises from the ambient environment to generate analog noise signals. The analog/digital converter converts the analog noise signals to digital noise signals. The gain management unit includes a sampling module, an obtaining module, a calculating module and a noise processing module. The sampling module samples the digital audio signals generated by the decoding unit to obtain a plurality of amplitude values of the digital audio signals sampled. The obtaining module obtains the default gain value of the gain control unit from the storage unit. The calculating module calculates an actual audio energy during a predetermined period time by the default gain value and the amplitude values sampled in the predetermined period time. The noise processing module calculates an ambient noise level according to the digital noise signals converted, obtaining a reference audio energy according to the ambient noise level, comparing the actual audio energy with the reference audio energy, and generating a hearing protection signal if the actual audio energy reaches the reference audio energy.

In another aspect of the invention, the audio processing apparatus includes a storage unit, a processing unit, a decoding unit, a digital/analog converter, a gain control unit, a sound output device, a microphone and an analog/digital converter. The storage unit stores a plurality of audio files and a default gain value. The processing unit fetches an audio file from the storage unit. The decoding unit decodes the audio file fetched to generate digital audio signals. The digital/analog converter converts the digital audio signals to analog audio signals. The gain control unit amplifies the analog audio signals converted according to the default gain value stored in the storage unit. The sound output device outputs sound corresponding to the analog audio signals amplified. The microphone collects ambient noises from the ambient environment to generate analog noise signals. The analog/digital converter converts the analog noise signals to digital noise signals. The processing unit includes a sampling module, an obtaining module, a calculating module and a noise processing module. The sampling module samples the digital audio signals generated by the decoding unit to obtain a plurality of amplitude values of the digital audio signals sampled. The obtaining module obtains the default gain value of the gain control unit from the storage unit. The calculating module calculates an actual audio energy during a predetermined period time by the default gain value and the amplitude values sampled in the predetermined period time. The noise processing module calculates an ambient noise level according to the digital noise signals converted, obtaining a reference audio energy according to the ambient noise level, comparing the actual audio energy with the reference audio energy, and generating a hearing protection signal if the actual audio energy reaches the reference audio energy.

Other systems, methods, features, and advantages will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a block diagram of a hardware infrastructure of an audio processing apparatus for hearing protection in an ambient environment in accordance with a first preferred embodiment of the present invention;

FIG. 2 is a schematic diagram of main function modules of a gain management unit of FIG. 1;

FIG. 3 is a common flowchart of playing an audio file;

FIG. 4 is a flowchart of a preferred audio processing method for hearing protection in ambient environment by utilizing the audio processing apparatus of FIG. 1 in according with the first preferred embodiment of the present invention;

FIG. 5 is a flowchart of a preferred audio processing method for hearing protection in ambient environment by utilizing the audio processing apparatus of FIG. 1 in according with a second preferred embodiment of the present invention;

FIG. 6 is a block diagram of a hardware infrastructure of an audio processing apparatus for hearing protection in an ambient environment in accordance with a third preferred embodiment of the present invention; and

FIG. 7 is a schematic diagram of main function modules of a processing unit of FIG. 6.

### DETAILED DESCRIPTION OF THE INVENTION

The audio processing apparatus of the present invention allows hearing protection while outputting audio signals in an ambient environment. The audio processing apparatus can be used in a car stereo system and other portable electric device such as an MP3, an electronic book, a mobile telephone, and so on. In the preferred embodiment, for simplicity, the audio processing apparatus incorporated in an MP3 is depicted. The following detailed description of the embodiments is made with reference to the attached drawings.

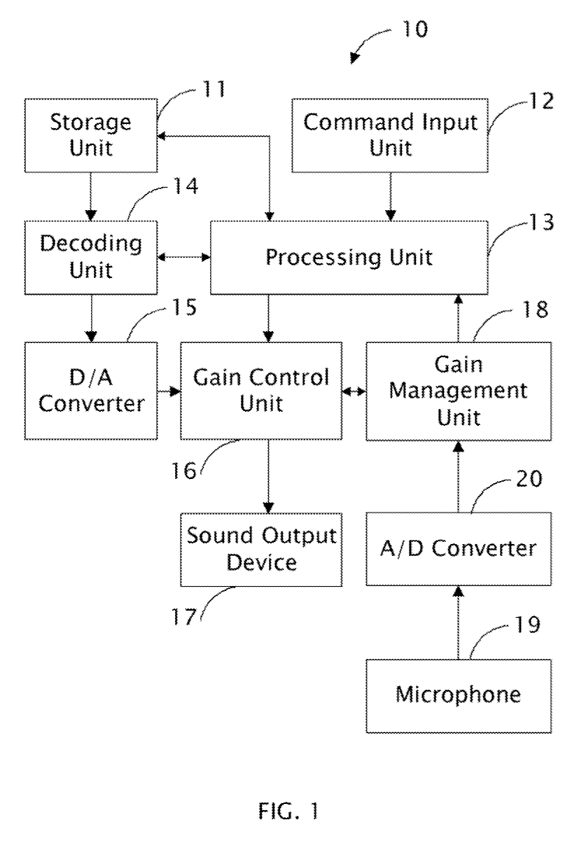

FIG. 1 is a block diagram of a hardware infrastructure of an audio processing apparatus 10 for hearing protection in an ambient environment in accordance with a first preferred embodiment of the present invention. The audio processing apparatus 10 includes a storage unit 11, a command input unit 12, a processing unit 13, a decoding unit 14, a digital/analog (D/A) converter 15, a gain control unit 16, a sound output device 17.

The storage unit 11 stores a plurality of audio files, a default gain value, and a decoding program. The storage unit 11 can be a flash storage, a hard disk driver, and the like. The processing unit 13 controls components of the apparatus 10, i.e., the storage unit 11, the command input unit 12, the decoding unit 14, and the gain control unit 16. The processing unit 13 can be a digital signal processor (DSP), a processing unit (CPU), and the like.

The command input unit 12 generates a command for playing an audio file in response to a selection operation on the audio file to be played. The processing unit 13 fetches the audio file from the storage unit 11 in response to the command for playing the audio file.

The decoding unit 14 activates the decoding program stored in the storage unit 11, decodes the audio file fetched from the storage unit 11 to generate digital audio signals. The D/A converter 15 converts the digital audio signals from the decoding unit 14 to generate analog audio signals.

The gain control unit 16 amplifies the analog audio signals received from the D/A converter 15 by a default gain value stored in the storage unit 11. The sound output device 17 outputs sound corresponding to the analog audio signals amplified. The sound output device 17 can be an earphone or a speaker.

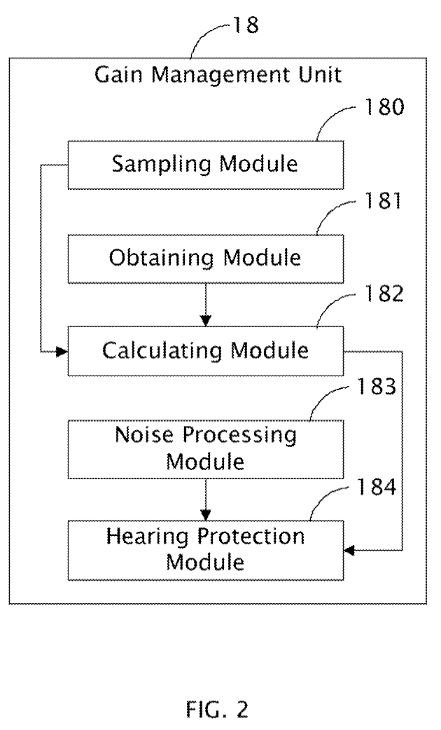

The audio processing apparatus 10 further includes a gain management unit 18 controlled by the processing unit 13. Referring to FIG. 2, the gain management unit 18 includes a sampling module 180, an obtaining module 181, a calculating module 182, a noise processing module 183, and a hearing protection module 184.

The sampling module 180 samples the digital audio signals generated by the decoding unit 14 to obtain a plurality of amplitude values of the digital audio signals sampled. The obtaining module 181 obtains the default gain value of the gain control unit 16 from the storage unit 11.

The calculating module 182 calculates an actual audio energy during a predetermined period time according to the default gain value and the amplitude values sampled in the predetermined period time by: Q=[Σ(mi*V)²/N]^{1/2}, where Q presents the actual audio energy, T presents the predetermined period time, i is any natural number, mi presents the amplitude values sampled in the predetermined period time T, V presents the default gain value, and N presents a count of the amplitude values mi sampled in the predetermined period time T.

The audio processing apparatus 10 further includes a microphone 19 and an analog/digital converter 20. The microphone 18 collects ambient noises from the ambient environment to generate analog noise signals. The analog/digital converter 20 converts the analog noise signals to digital noise signals.

The noise processing module 183 calculates an ambient noise level according to the digital noise signals generated, obtains a reference audio energy corresponding the ambient noise level calculated, compares the actual audio energy with the reference audio energy, and generates a hearing protection signal if the actual audio energy reaches the reference audio energy. The reference audio energy is an upper threshold allowable for a user's hearing.

The hearing protection module 184 signals the gain control unit 16 to automatically reduce the default gain value to a reduced gain value according to the hearing protection signal, and stores the reduced gain value in the storage unit 11, thus, updating the default gain value of the gain control unit 16 stored therein. Then, the gain control unit 16 amplifies the analog audio signals from the D/A converter 15 by the reduced gain value.

In other preferred embodiment, the hearing protection module 184 may generate prompt signals to prompt the user to manually reduce the default gain value to the reduced gain value according to the hearing protection signal, and stores the reduced gain value in the storage unit 11, thus, updating the default gain value of the gain control unit 16 stored therein. Then, the gain control unit 15 amplifies the prompt signals generated, and emits the prompt signals amplified to the sound output device 17. Furthermore, the hearing protection module 184 may also directly emit the prompt signals generated to the sound output device 17.

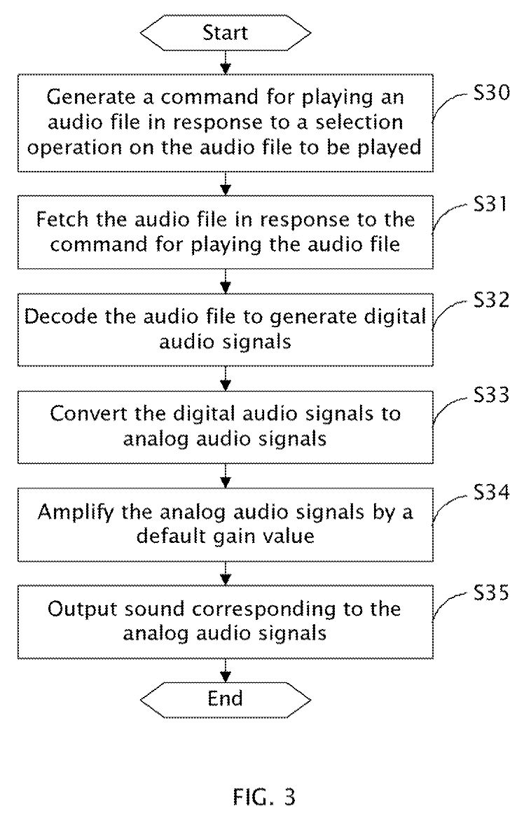

FIG. 3 is a common flowchart for playing an audio file. In step S30, the command input unit 12 generates the command for playing the audio file in response to the selection operation on the audio file to be played. In step S31, the processing unit 13 fetches the audio file from the storage unit 11 in response to the command generated by the command input unit 12.

In step S32, the decoding unit 14 decodes the audio file fetched from the storage unit 11 to generate digital audio signals. In step S33, the D/A converter 15 converts the digital audio signals from the decoding unit 14 to generate analog audio signals.

In step S34, the gain control unit 16 amplifies the analog audio signals from the D/A converter 15 by the default gain value stored in the storage unit 11. In step S35, the sound output device 17 outputs sound corresponding to the analog audio signals amplified.

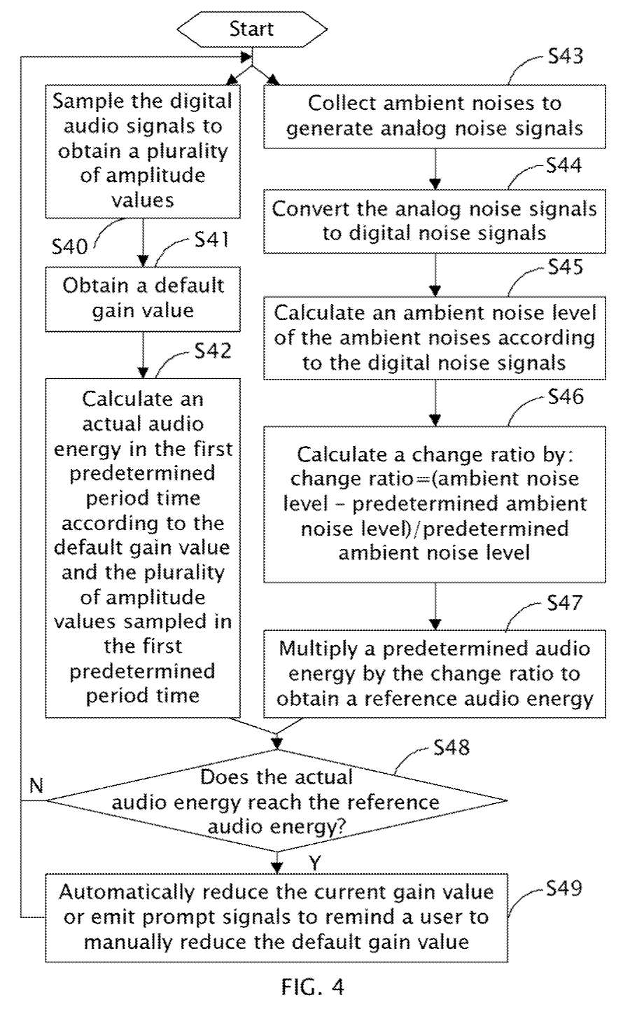

FIG. 4 is a flowchart of a first preferred audio processing method for hearing protection in ambient environment by utilizing the audio processing apparatus of FIG. 1. In step S40, the sampling module 180 samples the digital audio signals generated by the decoding unit 14 to obtain the plurality of amplitude values of the digital audio signals sampled. In step S41, the obtaining module 181 obtains the default gain value of the gain control unit 16 from the storage unit 11.

In step S42, the calculating module 182 calculates the actual audio energy during the predetermined period time according to the default gain value and the amplitude values sampled by the sampling module 180 in the predetermined period time.

Synchronously, in step S43, the microphone 18 collects ambient noises from the ambient environment. In step S44, the analog/digital converter 20 converts the ambient noises to the digital ambient noises.

In step S45, the noise processing module 183 calculates the ambient noise level according to the digital ambient noises converted by the analog/digital converter 20.

In step S46, the noise processing module 183 subtracts the predetermined ambient noise level from the ambient noise level to get the margin, divides the predetermined ambient noise level by the margin to obtain the change ratio by: change ratio=(ambient noise level−predetermined ambient noise level)/predetermined ambient noise level.

In step S47, the noise processing module 183 multiplies the predetermined audio energy by the change ratio to obtain the reference audio energy.

In step S48, the noise processing module 183 compares the actual audio energy with the reference audio energy, determines whether the actual audio energy reaches the reference audio energy. If the actual audio energy does not reach the reference audio energy, the procedure goes to start.

If the actual audio energy reaches the reference audio energy, in step S49, the hearing protection module 184 signals the gain control unit 16 to automatically reduce the default gain value to the reduced gain value according to the hearing protection signal, and stores the reduced gain value in the storage unit 11, thus, updating the default gain value stored therein, or emits prompt signals to prompt the user to manually reduce the default gain value according to the hearing protection signal, then the procedure goes to start.

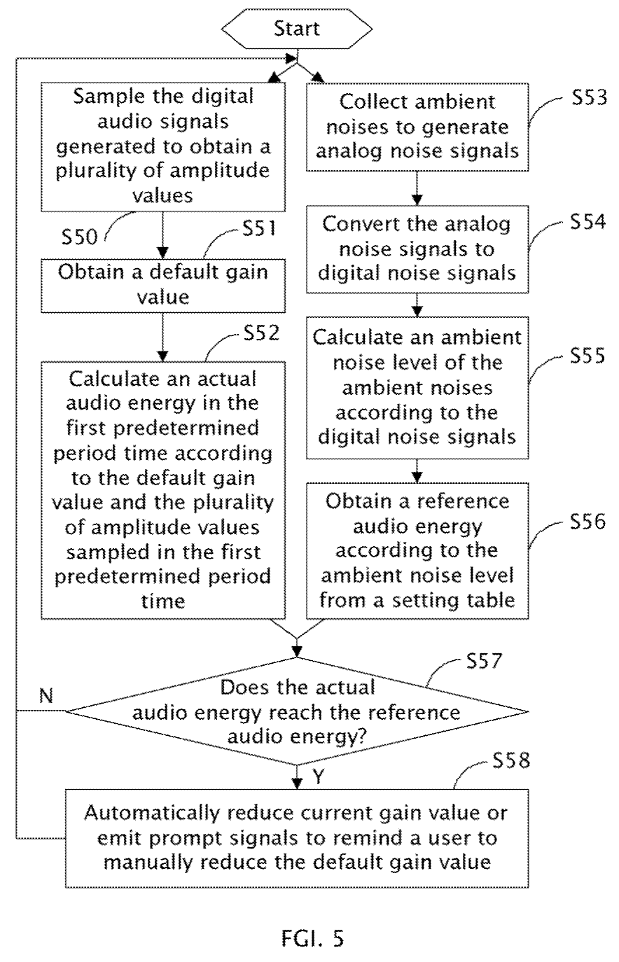

FIG. 5 is a flowchart of a second preferred audio processing method for hearing protection in ambient environment by utilizing the audio processing apparatus of FIG. 1. The difference between the second preferred method and the first preferred method is that in step S56, the noise processing module 183 obtains the reference audio energy corresponding the ambient noise level calculated from the setting table listed in the storage unit 11.

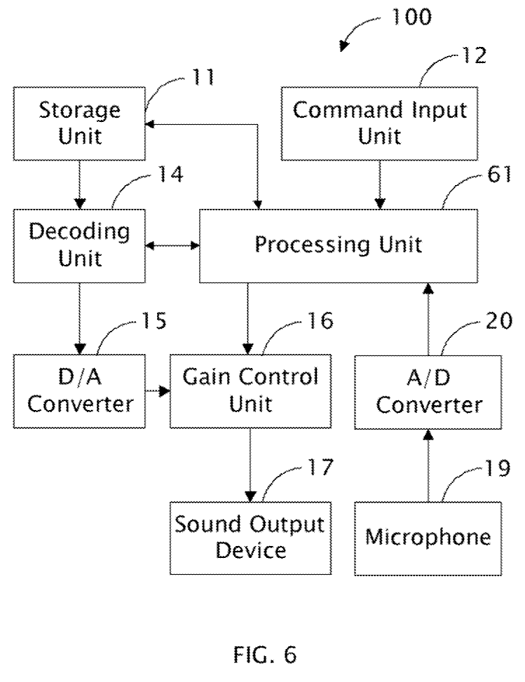

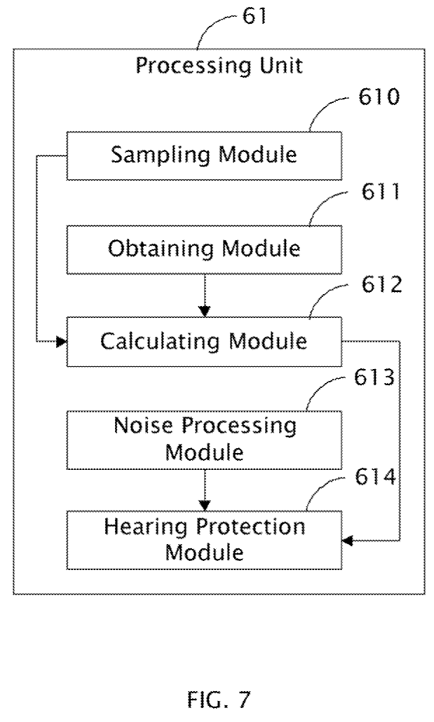

FIG. 6 is a block diagram of a hardware infrastructure of an audio processing system for hearing protection in accordance with a third preferred embodiment of the present invention. The difference between the third embodiment and the first and second embodiment is that the audio processing apparatus 100 of the third embodiment does not include the gain management unit 18, and a processing unit 61 performs the functions thereof. Referring to FIG. 7, the processing unit 61 includes a sampling module 610, an obtaining module 611, a calculating module 612, a noise processing module 613, and a hearing protection module 614, which respectively performs the same functions as the sampling module 180, the obtaining module 181, the calculating module 182, the noise processing module 183, and the hearing protection module 184 of the gain management unit 18 in the first and second embodiment.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. An audio processing method for hearing protection while an audio processing apparatus outputs audio signals in an ambient environment, the method comprising steps of:
sampling digital audio signals decoded to obtain a plurality of amplitude values of the digital audio signals;
obtaining a default gain value;
calculating an actual audio energy of the digital audio signals sampled during a predetermined period time according to the default gain value and the amplitude values sampled in the predetermined period time;
collecting ambient noises to calculate an ambient noise level;
obtaining a reference audio energy according to the ambient noise level;
comparing the actual audio energy with the reference audio energy; and
generating a hearing protection signal if the actual audio energy reaches the reference audio energy.

2. The audio processing method according to claim 1, wherein the step of obtaining the reference audio energy comprises the steps of:
providing a setting table for listing a plurality of reference audio energies corresponding to ambient noise levels; and
obtaining a reference audio energy corresponding the ambient noise level calculated from the setting table.

3. The audio processing method according to claim 1, wherein the step of obtaining the reference audio energy comprises the steps of:
subtracting a predetermined ambient noise level from the ambient noise level to get a margin;
dividing the predetermined ambient noise level by the margin to get a change ratio; and
multiplying a predetermined audio energy by the change ratio to obtain the reference audio energy.

4. The audio processing method according to claim 1, further comprising the steps of: automatically reducing the default gain value to a reduced gain value according to the hearing protection signal, and storing the reduced gain value to update the default gain value.

5. The audio processing method according to claim 1, further comprising the steps of: emitting prompt signals to remind a user to reduce the default gain value to a reduced gain value according to the hearing protection signal, and storing the reduced gain value to update the default gain value.

6. The audio processing method according to claim 1, wherein the actual audio energy is calculated by Q=[Σ(mi*V)²/N]^{1/2}, where Q presents the actual audio energy, T presents the predetermined period time, i is any natural number, mi presents the amplitude values sampled in the predetermined period time T, V presents the default gain value, and N presents a count of the amplitude values mi sampled in the predetermined period time T.

7. An audio processing apparatus for hearing protection in an ambient environment, the apparatus comprising:
a storage unit for storing a plurality of audio files and a default gain value;
a processing unit for fetching an audio file from the storage unit;
a decoding unit for decoding the audio file fetched to generate digital audio signals;
a digital/analog converter for converting the digital audio signals to analog audio signals;
a gain control unit for amplifying the analog audio signals converted according to the default gain value stored in the storage unit;
a sound output device for outputting sound corresponding to the analog audio signals amplified;
a microphone for collecting ambient noises from the ambient environment to generate analog noise signals;
an analog/digital converter for converting the analog noise signals to digital noise signals; and
a gain management unit comprising:
a sampling module for sampling the digital audio signals generated by the decoding unit to obtain a plurality of amplitude values of the digital audio signals sampled;
an obtaining module for obtaining the default gain value of the gain control unit from the storage unit;
a calculating module for calculating an actual audio energy during a predetermined period time by the default gain value and the amplitude values sampled in the predetermined period time; and
a noise processing module for calculating an ambient noise level according to the digital noise signals converted, obtaining a reference audio energy according to the ambient noise level, comparing the actual audio energy with the reference audio energy, and generating a hearing protection signal if the actual audio energy reaches the reference audio energy.

8. The audio processing apparatus according to claim 7, wherein the storage unit further comprises a setting table for listing a plurality of reference audio energies corresponding to ambient noise levels, and the noise processing module obtains the reference audio energy corresponding the ambient noise level from the setting table.

9. The audio processing apparatus according to claim 7, wherein the noise processing module subtracts a predetermined ambient noise level from the ambient noise level to get a margin, divides the predetermined ambient noise level by the margin to get a change ratio, and multiplies a predetermined audio energy by the change ratio to obtain the reference audio energy.

10. The audio processing apparatus according to claim 7, wherein the audio processing apparatus further comprises a hearing protection module for automatically reducing the default gain value to a reduced gain value according to the hearing protection signal, and storing the reduced gain value in the storage unit to update the default gain value stored therein.

11. The audio processing apparatus according to claim 7, wherein the audio processing apparatus further comprises a hearing protection module for emitting prompt signals to remind a user to reduce the default gain value to a reduced gain value according to the hearing protection signal, and storing the reduced gain value in the storage unit to update the default gain value stored therein.

12. The audio processing apparatus according to claim 7, wherein the actual audio energy is calculated by Q=[Σ(mi*V)²/N]^{1/2}, where Q presents the actual audio energy, T presents the predetermined period time, i is any natural number, mi presents the amplitude values sampled in the predetermined period time T, V presents the default gain value, and N presents a count of the amplitude values mi sampled in the predetermined period time T.

13. An audio processing apparatus for hearing protection in an ambient environment, the apparatus comprising:
a storage unit for storing a plurality of audio files and a default gain value;
a processing unit for fetching an audio file from the storage unit;
a decoding unit for decoding the audio file fetched to generate digital audio signals;
a digital/analog converter for converting the digital audio signals to analog audio signals;
a gain control unit for amplifying the analog audio signals converted according to the default gain value;
a sound output device for outputting sound corresponding to the analog audio signals amplified;
a microphone for collecting ambient noises from the ambient environment to generate analog noise signals;
an analog/digital converter for converting the analog noise signals to digital noise signals;
wherein the processing unit comprises:
a sampling module for sampling the digital audio signals generated by the decoding unit to obtain a plurality of amplitude values of the digital audio signals sampled;
an obtaining module for obtaining the default gain value of the gain control unit from the storage unit;
a calculating module for calculating an actual audio energy during a predetermined period time by the default gain value and the amplitude values sampled in the predetermined period time; and
a noise processing module for calculating an ambient noise level according to the digital noise signals converted, obtaining a reference audio energy according to the ambient noise level; comparing the actual audio energy with the reference audio energy, and generating a hearing protection signal if the actual audio energy reaches the reference audio energy.

14. The audio processing apparatus according to claim 13, wherein the storage unit further comprises a setting table for listing a plurality of reference audio energies corresponding to ambient noise levels; the noise processing module obtains the reference audio energy corresponding the ambient noise level calculated from the setting table.

15. The audio processing apparatus according to claim 13, wherein the noise processing module subtracts a predetermined ambient noise level from the ambient noise level to get a margin; divides the predetermined ambient noise level by the margin to get a change ratio; and multiplies a predetermined audio energy by the change ratio to obtain the reference audio energy.

16. The audio processing apparatus according to claim 13, wherein the audio processing apparatus further comprises a hearing protection module for automatically reducing the default gain value to a reduced gain value according to the hearing protection signal, and storing the reduced gain value in the storage unit to update the default gain value stored therein.

17. The audio processing apparatus according to claim 13, wherein the audio processing apparatus further comprises a hearing protection module for emitting prompt signals to remind a user to reduce the default gain value to a reduced gain value according to the hearing protection signal, and storing the reduced gain value in the storage unit to update the default gain value stored therein.

18. The audio processing apparatus according to claim 13, wherein the actual audio energy is calculated by Q=[Σ(mi*V)²/N]^{1/2}, where Q presents the actual audio energy, T presents the predetermined period time, i is any natural number, mi presents the amplitude values sampled in the predetermined period time T, V presents the default gain value, and N presents a count of the amplitude values mi sampled in the predetermined period time T.
